(19) United States
(12) Patent Application Publication
(10) Pub. No.: US 2021/0353160 A1
(43) Pub. Date: Nov. 18, 2021

(54) MATHEMATICAL BIOMARKER FOR ARTERIAL VISCOELASTICITY ASSESSMENT

(71) Applicant: KING ABDULLAH UNIVERSITY OF SCIENCE AND TECHNOLOGY, Thuwal (SA)

(72) Inventors: Taous Meriem LALEG, Thuwal (SA); Mohamed Abdelwaheb BAHLOUL, Thuwal (SA)

(21) Appl. No.: 17/284,072

(22) PCT Filed: Oct. 8, 2019
(86) PCT No.: PCT/IB2019/058566

§ 371 Date: Apr. 9, 2021

Related U.S. Application Data
(60) Provisional application No. 62/872,305, filed on Jul. 10, 2019.
(60) Provisional application No. 62/747,325, filed on Oct. 18, 2018.

(51) Int. Cl. A61B 5/02; A61B 5/021; A61B 5/026; A61B 5/00

(52) U.S. Cl. CPC A61B 5/02007; A61B 5/7239; A61B 5/026; A61B 5/021

(57) ABSTRACT

A method for assessing a state of a cardiovascular system includes receiving a blood pressure Pa and a blood flow Qa of the cardiovascular system; calculating with a fractional-order, viscoelastic Windkessel model an arterial compliance C_{α} of the cardiovascular system; and evaluating the state of the cardiovascular system based on a fractional-order parameter α associated with the fractional-order, viscoelastic Windkessel model.

### CROSS-REFERENCE TO RELATED APPLICATIONS

This application claims priority to U.S. Provisional Patent Application No. 62/747,325, filed on Oct. 18, 2018, and U.S. Provisional Patent Application No. 62/872,305, filed on Jul. 10, 2019, both entitled “MATHEMATICAL BIOMARKER FOR ARTERIAL VISCOELASTICITY ASSESSMENT,” the disclosures of which are incorporated herein by reference in their entirety.

### BACKGROUND

### Technical Field

Embodiments of the subject matter disclosed herein generally relate to arterial hemodynamic assessment, and more specifically, to a fractional differentiation model that predicts arterial parameters.

### Discussion of the Background

Arterial viscoelasticity assessment is very useful for the prevention and early diagnosis of a wide range of cardiovascular diseases, such as atherosclerosis. Accordingly, modeling the mechanical properties of the vessel wall tissue and the simulation of their effects on the arterial hemodynamic were considered as having a substantial role in clinical routine. Indeed, the characterization and emulation of the arterial viscoelasticity deepen our understanding of the onset vascular pathology and causes. In addition, they can serve as reliable diagnosis tools.

The arterial system is completely coupled to the heart such that the contractile state of the left ventricle and its produced central blood pressure (the pressure in the aorta) are in tune with the arterial mechanical properties. The interactions between the left ventricle and the systemic arteries are considered to govern an appropriate and normal cardiovascular function. In the past, numerous methods have been proposed to characterize the complex vascular after-load presented by the systemic arteries to the left ventricle, which are known as aortic input impedance models. In general, it is challenging to measure such vascular parameter directly. However, nowadays, blood pressure and flow waveforms at the arterial entrance (or alternatively aortic input impedance, which is expressed as the ratio of the blood pressure and flow in the frequency domain) are practically accessible.

Accordingly, the derivation of the arterial mechanical properties from measured blood pressure and flow at the entrance of the systemic system is possible. This is equivalent to solving a single equation with two known inputs: blood flow and pressure (the equivalent of the aortic input impedance) and multiple unknowns (mechanical parameters). A typical approach in solving this so called “hemodynamic inverse problem” is based on fitting the real input impedance to a reduced model and then the resulting estimated parameters represent the vascular properties.

Several reduced models have been proposed in this regard. The well-known Windkessel (WK) lumped parametric model has been considered, for a long time, as an acceptable approximation of the aortic input impedance. In fact, the WK models 1) comprise a reduced numbers of unknown parameters, 2) are able to fit the real input impedance at low and high frequency, and 3) involve physiologically interpretable elements. However, the WK models have various limitations such as the inability of presenting all the arterial mechanical properties of interest accurately, such as arterial stiffness.

The WK models are capable of characterizing the arterial system parameters, such as: 1) aortic characteristic impedance, 2) arterial compliance, and 3) arterial peripheral resistance. In general, the WK models can be classified into elastic and viscoelastic models. Elastic WK models consider the mechanical behavior of the vessel's wall as purely elastic and represented by an ideal capacitor. However, the viscoelastic Windkessel (VWK) models assume that the blood vessel exhibit both elastic and viscous properties, which is consistent with several studies that have claimed that, similar to most biological tissues, the CVS wall material shows a viscoelastic behavior rather than pure elastic behavior.

Indeed, arteries are considered as a viscoelastic reservoir. They are modeled as a superposition of viscous and elastic components where the transmission of the mechanical energy might be split into two modules: the first module represents the stored energy into the wall tissues, which can be retrieved due to the elasticity capabilities, while the second module refers to the dissipated energy caused by the viscous property. Thus, an understanding of the blood vessel viscoelasticity and the knowledge of its variety effect are necessary for understanding the degenerative diseases that affect the arterial network.

Recently, it has been shown that the arterial wall motion can be described using fractional-order models consisting of fractional differential equations, thus reducing the number of parameters and showing a natural response [1]-[5]. These fractional-order models have interesting properties that help in more accurately modeling complex systems, including biological systems.

Thus, there is a need to apply such a model that better describes the arterial viscoelasticity of humans for the prevention and early diagnosis of a wide range of cardiovascular diseases.

### SUMMARY

According to an embodiment, there is a method for assessing a state of a cardiovascular system. The method includes receiving a blood pressure Pa and a blood flow Qa of the cardiovascular system; calculating with a fractional-order, viscoelastic Windkessel model an arterial compliance C_{α} of the cardiovascular system; and evaluating the state of the cardiovascular system based on a fractional-order parameter α associated with the fractional-order, viscoelastic Windkessel model.

According to another embodiment, there is a computing device for assessing a state of a cardiovascular system, and the computing device includes an interface for receiving a blood pressure Pa and a blood flow Qa of the cardiovascular system; and a processor connected to the interface. The processor is configured to calculate with a fractional-order, viscoelastic, Windkessel model an arterial compliance C_{α} of the cardiovascular system; and evaluate the state of the cardiovascular system based on a fractional-order parameter α associated with the fractional-order, viscoelastic Windkessel model.

According to still another embodiment, there is a non-transitory computer readable medium including computer executable instructions, wherein the instructions, when executed by a processor, implement instructions for assessing a state of a cardiovascular system as discussed above.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 is a schematic illustration of an electrical circuit that represents the WK model;

FIG. 2A illustrates the electrical circuit of the viscoelastic WK model having a complex capacitor, and FIG. 2B illustrates the electrical circuit of the viscoelastic WK model having a ladder network;

FIG. 3 illustrates a fractional-order capacitor and its equivalent electrical circuit;

FIG. 4 illustrates a fractional-order WK model;

FIGS. 5A to 5D illustrate the distribution, mean value and standard deviation in (mmHg) of (1) the mean blood pressure (MBP), (2) the pulse pressure (PP), (3) diastolic pressure (DP), and (4) systolic pressure (SP) at the level of ascending aorta for the in-silico data-base;

FIG. 6 is a flowchart of a method for assessing the state of the arterial system in a patient;

FIG. 7A is a table showing various parameters calculated for the novel model and traditional models, and FIG. 7B presents box plots that illustrate summary statistics of the two- and three-elements novel models for various values of a fractional-order parameter;

FIG. 8 illustrates the RMSE for the novel models and traditional models;

FIG. 9 illustrates the deviation, in percentage, for the novel models and the traditional models;

FIG. 10 illustrates the NMSE of the phase angle for the novel models and the traditional models;

FIG. 11 illustrates the effective compliance for the novel models and the traditional models;

FIG. 12 illustrates the effective compliance for a three-elements novel model and a corresponding three-elements traditional model;

FIG. 13 illustrates the characteristic impedance for the three-elements novel model and a corresponding three-elements traditional model;

FIGS. 14A to 14F illustrate the blood pressure (BP) and flow (BF) at the level of the ascending aorta in time domain;

FIGS. 15A to 15F illustrate the aortic input impedance modulus (presented in log-scale) and the phase angle as a function of frequency and a comparison between the reconstructed impedance modulus and phase angle, based on the two-elements WK model and a novel two-elements model;

FIG. 16 is a flowchart of a method for assessing a state of a cardiovascular system; and

FIG. 17 is a schematic diagram of a computing device that implements the novel methods discussed herein.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. For simplicity, the following embodiments are discussed with regard to the modeling of the arterial viscoelasticity in humans. However, the methods and systems discussed herein are equally applicable to other organism that have viscoelastic pipes that can be modeled with a fractional-order capacitor.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, there is a model that is based on the elements: the aortic characteristic impedance Z_{C}, a fractional-order capacitor (FoC) C_{F} (or Constant phase Element), and the total peripheral resistance Rₚ. The ideal capacitor of the 3-WK model is substituted in this method by the fractional-order capacitor C_{F}. The latter non-ideal component combines both resistive and capacitive properties, which displays the fractional viscoelastic behavior of the arterial vessel. The contribution of both properties is controlled by the fractional differentiation order a, enabling thus an accurate and reliable physiological description. The effect of varying the fractional differentiation order value a on the complex and frequency dependent arterial compliance C_{α} and arterial stiffness AS is discussed later. In addition, the effect of the a values on the modulus and phase angle of the aortic input impedance is also investigated.

Before discussing the novel fractional-order viscoelastic (FWK) model, a short review of the traditional WK model is believed to be in order. The WK lumped parameter model is the most commonly used 0-Dimension method (No space variation) that describes the arterial system as a function of two- or three-elementary analog device components: a resistor, a capacitor, and an inductor. WK models describe the arterial hemodynamic by linking the blood flow, and blood pressure to the arterial resistance and compliance. This link is formulated based on the analogy between physiological, hydraulic and electrical systems.

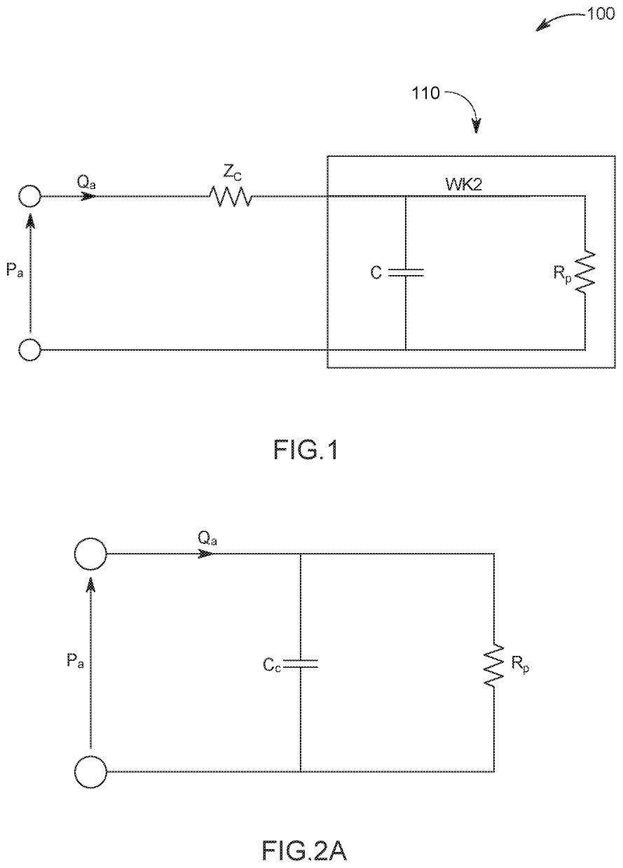

The two-elements WK model (WK2 herein) includes a resistor, which represents the total arterial peripheral resistance, and a capacitor, which is connected in parallel to the resistor. The WK2 model is shown in FIG. 1 as block 110. The capacitor is an ideal capacitor, which characterizes the total vessel compliance. While the WK2 is very simple, it does not produce the real systemic input impedance and fails to predict the aortic pressure in the systolic phase. In fact, the frequency analysis of the aortic input impedance pattern shows that the complex modulus decreases to a negligible value and the phase angle reaches −90° at medium and high frequencies.

This response is in contrast to that measured in-vivo, where the modulus reduces to a plateau value and the phase angle tends to 0°. In order to overcome this weakness, a three-element WK model (WK3) has been proposed. Compared to the WK2 model, the WK3 model 100 adds a characteristic impedance Z_{C}, which is connected in series to the WK2 block 110, as shown in FIG. 1. The characteristic impedance Z_{C} helps to improve the frequency response and provides a realistic shape for the blood flow and aortic pressure.

Since the original model was introduced, several modified WK configurations have been suggested with the objective to improve the accuracy of the arterial hemodynamic prediction. These models vary in the numbers of electrical elements used to represent the arterial system variables. Although, the WK3 model and the latest WK modified models were able to produce an acceptable hemodynamic characterization, there are indications that the compliance C (described by the ideal capacitor), which represents the elasticity ability of the blood vessel, is not well estimated by these models. Indeed, these models considered that the arterial vessel is a pure elastic reservoir (represented by an ideal capacitor), which contrasts the natural behavior of the vessels.

In fact, similar to the most existing biological tissues, the vascular vessel wall is characterized by a viscoelastic behavior rather than a pure elastic one. Thus, the transmission of the mechanical energy through the arteries might be split into two modules: a first module that represents the stored energy into the wall tissues that can be retrieved due to the elasticity capabilities, and a second module that represents the energy that is dissipated due to the viscosity property. Based on this idea, some models tried to characterize this behavior by developing the viscoelastic Windkessel (VWK) models. Those representations are similar to the standard WK.

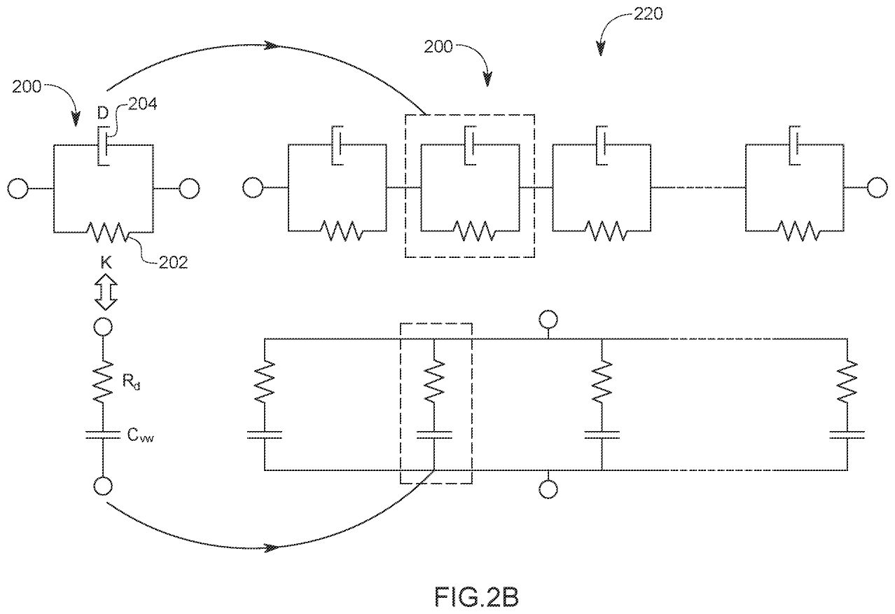

However, the ideal capacitor C in the WK model is replaced by a complex and frequency dependent capacitor in the VWK model. The complex compliance in the VWK model is developed based on the mechanical Voigt cell model 200 shown in FIG. 2A. The Voigt cell connects a lossless elastic element (spring) 202 and a lossy viscous damper (dashpot) 204 to represent respectively the elastic (often Hookean) and viscous (often Newtonian) properties of the bio-tissue. The Voigt mechanical cell (a spring connected in parallel to a dashpot), as shown in FIG. 2A, is the elementary model that can describe the viscoelastic behavior of such bio-mechanics collagenous tissue. The electrical analogous of the Voigt mechanical cell 200 is a resistor R_{d} connected in series to a capacitor C_{vw}, as shown in FIG. 2A. The resistor R_{d} and the capacitor C_{vw} represent the viscous and elastic phenomena. Based on the Voigt cell, the ideal constant capacitor C accounting for the total arterial compliance in the WK model is now substituted by a complex and frequency dependent capacitor C_{c}(jω). This capacitor C_{c} in FIG. 2A corresponds to the electrical analogy of the standard Voigt cell. It comprises the resistor R_{d} in serial with a capacitor C_{vw}, which represents the viscous losses and static compliance, respectively. The complex and frequency dependent compliance C_{c}(jω) can be expressed as follow:

$${C}_{c} ( j ω ) = {C}_{v w} \frac{1}{1 + j ω {R}_{d} {C}_{v w}} . \qquad ( 1 )$$

However, it was shown that modeling the arterial vessel motion using the Voigt cell model is limited and does not yield a realistic representation of the vascular viscoelasticity. This is because the Voigt characterization does not account for the stress-relaxation experiment.

This issue was believed to be solved by increasing the viscoelastic model order, as illustrated by the ladder network 220 in FIG. 2B. However, with the ladder model 220, the number of parameters becomes very large, which introduces another challenge. The physiological aortic input impedance, which is based on the collected data, is deemed insufficient to identify all the variables for these complex models. In addition, it is desired that reduced order models are used for their simplicity and their ease to characterize the arterial system.

Recent studies have indicated that the arterial viscoelasticity exhibits a fractional order behavior. Accordingly, the study of the vascular wall motion within the fractional modeling framework may overcome the previously mentioned inconsistencies. In fact, fractional order models may provide good behavior with less parameters. In addition, the representation of the arterial viscoelasticity using the ladder structure can be simply be replaced by a two-element fractional order model, which is able to accurately capture the real behavior of the arterial vessels.

Mechanical fractional-order viscoelasticity models (FVM) such as Voigt-FVM and Standard-Linear-Solid-FVM have been used to investigate the effect of a hemodynamic index e.g., heart rate (HR) on arterial viscoelasticity. In general, a FVM model includes a pure spring and one or two fractional-order mechanical components, the spring-pots. In this model, the fractional element displays the fractional-order derivative relationship between the mechanical stress σ(t) and strain ϵ(t) on the vessel, as described in the following equation:

σ(t)=ηDₜ^{α}ϵ(t), (2)

where α is the fractional differentiation order parameter that controls the level of viscoelasticity of the artery and η is a constant of proportionality. As α approaches the value 1, the artery's behavior is similar to a pure viscous dashpot (more resistive) and when α borders 0, the vessel wall motion is more like a pure elastic spring.

However, even this model does not describe with a sufficient degree of accuracy the characteristics of the arterial systems. Thus, in the following embodiments, a fractional-order WK (FWK) model is introduced. This model relies on the fractional derivative (FD). The FD is a generalization to a non-integer order of the traditional integer derivative. Due to its non-locality and memory properties, FD is a powerful tool for modeling physical phenomena involving memory effect or delays. The differentiation orders offer new parameters that allow capturing more general behaviors of the system that is investigated, than the integer order models.

The biomechanical behavior of the arteries is determined by its three main wall components: smooth muscle fibers, elastin fibers, and collagen fibers. Vascular activation is defined as the smooth muscle fiber stretching action on the collagenous fiber. Scientists have noticed that this activity affects the local viscoelastic behavior of the vessels. Recently, there were some proposals [2]-[4] to apply a fractional order model to describe the response of the aorta viscoelasticity. These studies showed that the fractional differentiation order value (α) can be associated with the arterial modulating viscoelasticity, smooth muscles action. In order to validate this model, the stress-relaxation experiment has been used.

The fractional-order derivative (FD) is a generalization of the traditional integer derivative to a non-integer order. The integral and the differential operators could be expressed as one unified (differ-integration) operator Dₜ^{α}, which is defined as follows:

$${D}_{t}^{α} = \{ \begin{matrix}\frac{{d}^{α}}{{dt}^{α}} & if α > 0 \\ 1 & if α = 0 \\ {\int }_{t}^{0} {( df )}^{- α} & if α < 0\end{matrix} \qquad ( 3 )$$

where α is an arbitrary real order of the operator (integral or derivative) known as the fractional order and df is the derivative function. Numerous fractional calculus definitions are possible. These definitions can be classified into two main classes. In the first class, the operator Dₜ^{α} is converted into the standard differential-integral operator when α is integer.

For instance, according to Reimann and Liouville definition, the fractional order derivative α of a function g(t) can be formulated as:

$${D}^{α} g ( t ) = \frac{1}{Γ ( 1 - α )} \frac{d}{d t} {\int }_{0}^{t} \frac{g ( τ )}{{( 1 - τ )}^{α}} d τ , \qquad ( 4 )$$

where Γ is the Euler gamma function, t is the time, and τ is a variable that is integrated from zero to t. The second class is the Laplace transform of Dₜ^{α}, which is s^{α}, assuming null initial conditions. Thus, the fractional operator in the Laplace domain becomes:

$${D}^{α} g ( t ) {\to }^{L} {s}^{α} G ( s ) , \qquad ( 5 )$$

where s is the Laplace variable. The Fourier transform of such operator can be found by substituting s by jω and thus, the equivalent frequency-domain expression of s^{α} are:

$$\begin{matrix}{( j ω )}^{α} = {ω}^{α} ( cos \frac{a π}{2} - j sin \frac{a π}{2} ) , & ( 6 ) \\ \frac{1}{{( j ω )}^{α}} = \frac{1}{{ω}^{α}} ( cos \frac{a π}{2} + j sin \frac{α π}{2} ) . & ( 7 )\end{matrix}$$

The fractional order derivative is now used in a systemic arterial system. A lumped parametric model is an analog circuit arrangement that leads to the same input impedance as the arterial network, in a specific frequency domain. The novel model has a similar configuration as the WK3 model, but incorporates a fractional-order capacitor rather than an ideal capacitor. The non-ideal capacitor represents the fractional arterial viscoelasticity behavior.

A fractional order capacitor (FoC) is an electrical component which consists of two parallel plates confining a lossy material. A lossy material is defined herein as a material that uses up electrical energy. In other words, an ideal capacitor has two parallel plates that confines a dielectric material. A dielectric material does not use electrical energy. This means that if an ideal capacitor is charged with a given charge, the ideal capacitor stores the energy associated with those charges. However, a fractional order capacitor charged with the same energy slowly consumes that energy. A fractional order capacitor can also be defined as a constant phase element (CPE) whose equivalent impedance has a constant phase angle (between 0° and ˜90° over the entire frequency band, from zero to infinity.

The relationship linking the current i_{c} flowing through an ideal capacitor C and the voltage v_{c} between its terminals is as follow:

$${i}_{c} = C \frac{d {v}_{c}}{d t} . \qquad ( 8 )$$

Applying the Laplace transformation to equation (8), and assuming again null initial conditions, the following equation is obtained:

I_{c}=CsV_{c}, (9)

The impedance of an ideal capacitor is the ratio of the voltage to the current flowing, in the frequency domain. Accordingly, the capacitor's impedance in the Laplace domain can be expressed as:

$${Z}_{c} ( s ) = \frac{1}{C s} . \qquad ( 10 )$$

The relation between the current and voltage of the fractional-order capacitor in the time domain is given by:

$${i}_{c} ( t ) = \frac{1}{{A}_{α}} {D}_{t}^{α} {v}_{c} ( t ) , \qquad ( 11 )$$

where A_{α} is the coefficient of the pseudo capacitance expressed in units of Farad·sec⁻¹. Applying the Laplace transform as defined in equations (5) to (7), and assuming null initial conditions, the following expression is obtained for the impedance of the fractional-order capacitor:

$${Z}_{F} ( s ) = \frac{{V}_{c} ( s )}{{I}_{c} ( s )} = {A}_{α} {s}^{- α} . \qquad ( 12 )$$

The fractional impedance Z_{F} varies with the exponent a (0<α<1), which is the fractional differentiation order.

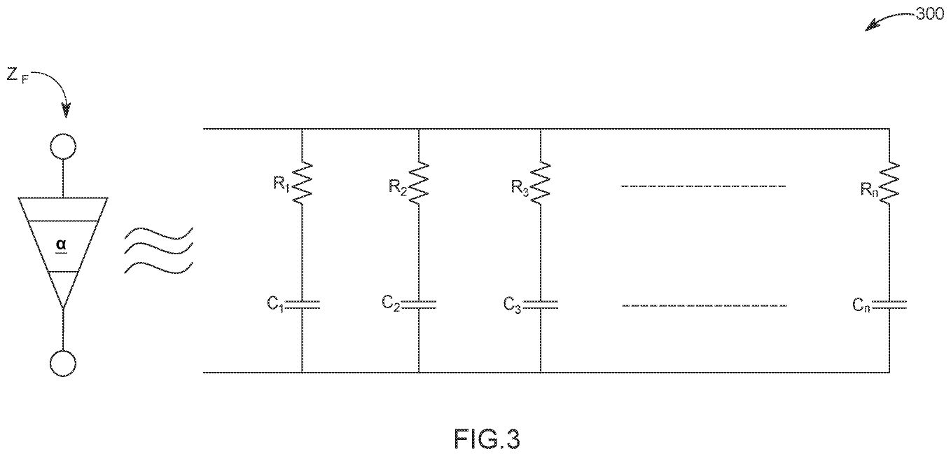

The fractional order impedance Z_{F} approximation noted in equation (12) might be implemented using an electrical circuit with a recursive association of resistance and capacitance elements. For example, as shown in FIG. 3, a Foster integer-order ladder RC network 300 may be used. This configuration is similar to the viscoelastic analog circuit model 220 illustrated in FIG. 2B. Furthermore, for s=jω, the value of the fractional capacitor at a specific frequency CO can be calculated using the expression:

$${Z}_{F} ( j ω ) = \underbrace{( {A}_{α} {ω}^{- α} cos ϕ )}_{{Z}_{D}} + \underbrace{{jA}_{α} {ω}^{- α} sin ϕ}_{{Z}_{S}} , \qquad ( 13 )$$

where the phase

$$ϕ = α \frac{π}{2} .$$

For α=0, Z_{F} refers to an ideal resistor whose phase angle equals 0°. For α=1, Z_{F} represents an ideal capacitor with a phase angle equal to −90°. The modulus of the fractional order impedance Z_{F} is given by:

$$\vert {Z}_{F} \vert = \sqrt{{( {A}_{α} {ω}^{- α} cos ( α \frac{π}{2} ) )}^{2} + {j ( {A}_{α} {ω}^{- α} sin ( α \frac{π}{2} ) )}^{2} .} \qquad ( 14 )$$

The fractional order impedance Z_{F} may be split into a dissipative component Z_{D} (represented by the real part) and a storage component Z_{S} (represented by the imaginary part). With analogy to the mechanical behavior of the arteries, the terms Z_{S} and Z_{D} correspond to the viscous and elastic parts of the arterial vessel wall. Thus, in this model, the fractional capacitor has both resistive and capacitive properties, and thus, the fractional capacitor represents the fractional viscoelastic behavior of the arterial vessel. The impact of both properties is coordinated by the fractional factor α, allowing a real physiological description of the arteries and decreasing the number of variables necessary to describe the arteries.

The fractional capacitor is used to describe the arteries as follows. The aortic input impedance Zᵢₙ is considered to characterize the systemic arterial system. Note that the aortic input impedance Zᵢₙ is different from the aortic characteristic impedance Z_{c} introduced in the model 100 in FIG. 1. The aortic input impedance Zᵢₙ represents the arterial parameters in a comprehensive way. The aortic input impedance Zᵢₙ represents a source of phenomenological information which depends just on the geometrical and mechanical characteristic of the arterial tree and the blood it encloses. Zᵢₙ is defined as a linear time invariant transfer function that relates the arterial blood pressure (Pa) to the blood flow (Qa) in the frequency domain. Thus, the aortic input impedance Zᵢₙ is defined as the ratio between (1) the frequency component of the output arterial blood pressure (Pa), and (2) the input blood flow (Qa) harmonics.

The blood pressure Pa is considered to be the central blood pressure at the level of aorta and it has a dependency on the properties of both the arterial system tree and the heart. Zᵢₙ is a complex function that comprises both real and imaginary parts. The hemodynamic analyses are usually based on the magnitude and phase of Zᵢₙ. The quantification of the arterial network energy dissipation is described by the real value of Zᵢₙ. When the aortic input impedance Zᵢₙ is known, a given blood flow Qa permits the control of the arterial pressure Pa and vice versa.

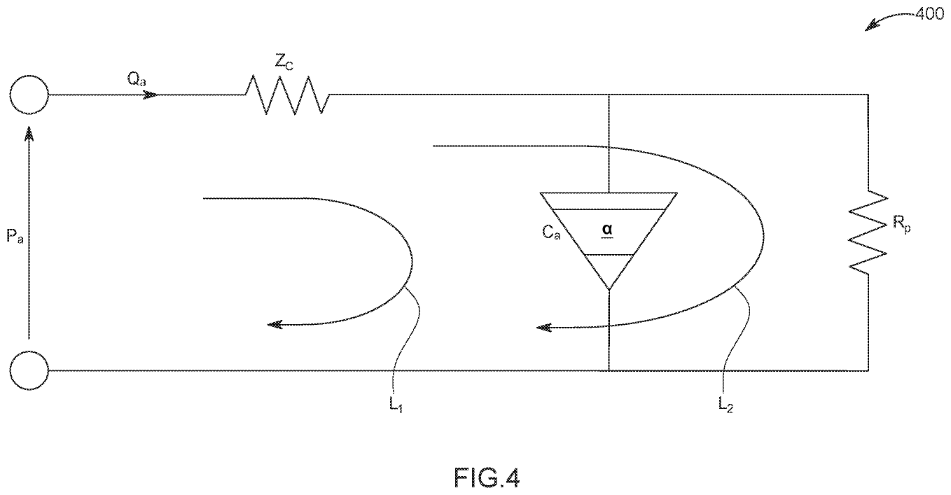

The model 400 that uses the fractional order capacitor is illustrated in FIG. 4 as an analogy with an electrical circuit that is made up of three elements: a fractional order capacitor C_{α}, the resistor Rp, and the characteristic impedance Z_{c}. This is the three-parameter FWK model, called herein the FWK3 model. If the characteristic impedance Z_{c} is selected to be zero, then a two-parameters FWK model is obtained, which is called herein the FWK2 model. The blood flow Qa corresponds to the current flowing through the electrical circuit and the arterial pressure Pa corresponds to the voltage applied to the electrical circuit. These quantities are governed by the Kirchhoff's Voltage Law (KVL) and Kirchhoff's Current Law (KCL) as now discussed.

If the KVL is applied to the first loop L1 in FIG. 4, the voltages along this loop can be written as:

V_{C}_{_{α}}(t)=Pₐ(t)−Z_{C}Qₐ(t) (15)

If the KCL is applied to the second loop L2 in FIG. 4, the current (i.e., the blood flow) along this loop can be written as:

$${Q}_{a} ( t ) = {C}_{α} {D}_{t}^{α} {V}_{{c}_{α}} ( t ) + \frac{{V}_{{c}_{α}} ( t )}{{R}_{p}} . \qquad ( 16 )$$

If equation (15) is substituted into equation (16), the following equation is obtained:

$${Q}_{a} ( t ) = {C}_{α} {D}_{t}^{α} ( {P}_{a} ( t ) - {Z}_{C} {Q}_{a} ( t ) ) + \frac{{P}_{a} ( t ) - {Z}_{C} {Q}_{a} ( t )}{{R}_{p}} . \qquad ( 17 )$$

Equation (17) can be rewritten as:

$${Z}_{C} {C}_{α} {D}_{t}^{α} {Q}_{a} ( t ) + {Q}_{a} ( t ) ( 1 + \frac{{Z}_{C}}{{R}_{p}} ) = {C}_{α} {D}_{t}^{α} {P}_{a} ( t ) + \frac{{P}_{a} ( t )}{{R}_{p}} . \qquad ( 18 )$$

If the Laplace transform is applied to equation (18) and the null conditions are assumed, the fractional aortic input impedance Z₃^{α} may be expressed as:

$$\begin{matrix}{Z}_{3}^{α} ( s ) = {Z}_{C} + \frac{{R}_{p}}{1 + {( {τ}_{α} s )}^{α}} , \\ or & ( 19 ) \\ {Z}_{3}^{α} ( s ) = ( {R}_{p} + {Z}_{C} ) \frac{1 + {( {τ}_{α N} s )}^{α}}{1 + {( {τ}_{α D} s )}^{α}} , & ( 20 )\end{matrix}$$

where the parameters τ_{N} and τ_{D} are time constants and have the following expressions:

$$\{ \begin{matrix}{τ}_{N} = \sqrt[α]{\frac{{R}_{p} {Z}_{C}}{{R}_{p} + {Z}_{C}} {C}_{α}} , \\ {τ}_{D} = \sqrt[α]{{R}_{p} {C}_{α}} .\end{matrix} \qquad ( 21 )$$

Note that the fractional aortic input impedance Z₃^{α} corresponds to the aortic input impedance Zᵢₙ defined above as the ratio of the blood pressure and the blood flow.

The model 400 illustrated in FIG. 4 and discussed above takes into consideration the fractional-order parameter α. The effects of varying the fractional differentiation order value a on the complex and frequency dependent arterial compliance C_{α} and arterial stiffness are now discussed. The variation of the aortic input impedance modulus |Z_{F}| and phase angle ∠Z_{F} for the model 400 versus a can be expressed as follow:

$$\begin{matrix}\vert {Z}_{3}^{α} \vert = ( {R}_{p} + {Z}_{C} ) \frac{\sqrt{{( 1 + {( ω {τ}_{N} )}^{α} cos ( a \frac{π}{2} ) )}^{2} + {( {( ω {τ}_{N} )}^{α} sin ( a \frac{π}{2} ) )}^{2}}}{\sqrt{{( 1 + {( ω {τ}_{D} )}^{α} cos ( a \frac{π}{2} ) )}^{2} + {( {( ω {τ}_{D} )}^{α} sin ( a \frac{π}{2} ) )}^{2}}} , and & ( 22 ) \\ {∠Z}_{3}^{α} = {tan}^{- 1} ( \frac{{( ω {τ}_{N} )}^{α} sin ( a \frac{π}{2} )}{1 + {( ω {τ}_{N} )}^{α} cos ( a \frac{π}{2} )} ) - {tan}^{- 1} ( \frac{{( ω {τ}_{D} )}^{α} sin ( a \frac{π}{2} )}{1 + {( ω {τ}_{D} )}^{α} cos ( a \frac{π}{2} )} ) . & ( 23 )\end{matrix}$$

The arterial stiffness (AS) is a metric used for the evaluation of the degenerative diseases physio-pathological mechanisms that affect CVS. AS characterizes the vessel material properties and is measured based on the Young's Modulus E. In clinical practice, it is not possible to directly measure E because isolating the arterial tissue without perfusion is not feasible. Hence, the pressure-volume and pressure-radius blood vessel relationships are used and these relationships define the total complex and frequency dependent arterial Compliance (C_{α}) and Elastance (E_{α}). These two metrics define the aptitude of the blood vessel to contract and expand with the variation of the blood pressure. The Arterial Compliance C_{α} is the inverse of Ea and they can be expressed as follow:

$${C}_{α} = \frac{1}{{E}_{α}} = \frac{Δ V}{Δ P} . \qquad ( 24 )$$

To estimate the Young Modulus Eₐ, the following relation that links E_{α} to C_{α} may be used:

$${C}_{α} \approx \frac{3 π {lr}_{i}^{2}}{2 {E}_{α} h} , \qquad ( 25 )$$

where rᵢ is the internal artery radius, h is the vessel wall thickness, and I is the arterial length.

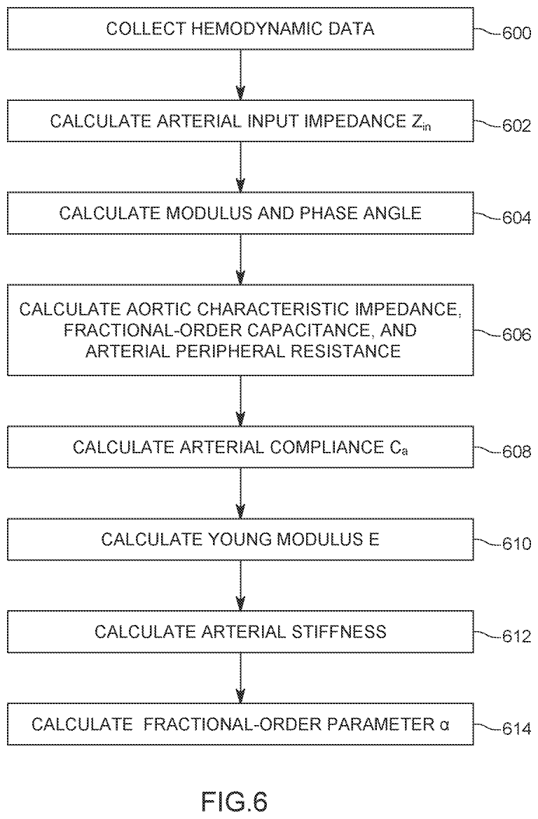

The equations discussed above can now be used with the model 400 to characterize the arterial stiffness of a patient. A method for using this model is discussed with regard to FIG. 6. In step 600, hemodynamic data is collected. The hemodynamic data may include blood pressure Pa and blood flow Qa. In one application, the hemodynamic data may also include a cardiac output. The blood pressure may be measured with any known device, for example, with a blood pressure monitor. The blood flow can be measured with ultrasonic or electromagnetic sensors, for example, a Doppler flowmeter. While in a typical clinical situation, only the modulus of the blood pressure and the blood flow are measured, in one application, both the modulus and the phase angle of the blood pressure and the blood flow can be measured.

In step 602, the blood pressure Pa and the blood flow Qa are used to calculate, the arterial input impedance Zᵢₙ. In step 604, the modulus and the phase angle of the arterial input impedance Zᵢₙ are calculated (see equations (22) and (23)). In step 606, the aortic characteristic impedance Z_{C}, the fractional-order capacitance C_{F}, and the arterial peripheral resistance Rₚ are calculated from the modulus |Z_{F}| and the phase angle ∠Z_{F} of the fractional-order arterial input impedance Z_{F}.

In step 608, the arterial compliance C_{α} is calculated so that the arterial compliance C_{α} is the fractional-order impedance Z_{F} of the fractional-order capacitor C (see equation (19)). In step 610, the Young modulus E is calculated based on equations (24 and 25), from the compliance C_{α}. Then, in step 612, the arterial stiffness AS is calculated based on the Young modulus E_{α}. In step 614, the fractional-order parameter α can be determined. For example, in one application, blood pressures and blood flows are collected from plural patients that have a known state of their cardiovascular system. The model 400 is run with these parameters and various fractional-order parameters α are applied until the curves of the three parameters of the model 400 fit the measured data of these patients. Then, the values of fractional-order parameters α that fit these curves are identified and ranges are established for this parameter. This means, as a way of an example, that for fractional-order parameter α smaller than 0.3 the patient may be hypotensive, for the fractional-order parameter α being between 0.3 and 0.7, the patient has a normal arterial system, and for the fractional-order parameter α being larger than 0.7, the patient is hypertensive. In other words, the model 400 can be calibrated based on known and accurate values of the arterial system and then, based only on the measured blood pressure and blood flow of a given patient, the model can produce a corresponding fractional-order parameter α that indicates the status of its cardiovascular system. The fractional-order parameter α can be used as a score for indicating the health of the patient. The numbers provided above are exemplary.

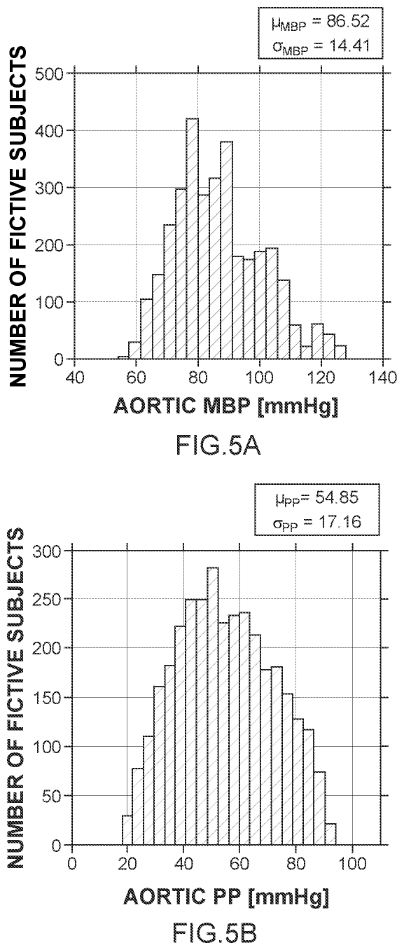

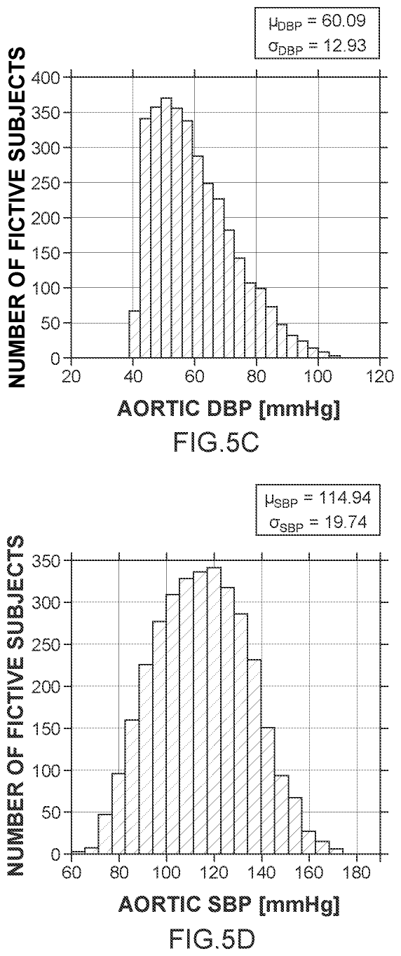

To test the model 400, virtual blood pressure and flow waveforms at the level of the ascending aorta have been used. This data was taken from a database generated in-silico from a validated one-dimensional numerical model of the arterial hemodynamics, whose cardiac and arterial parameters were varied within physiological ranges. The database includes 3,325 virtual healthy adult subjects. The model 400 is able to generate the major hemodynamic properties sensed in-vivo. FIGS. 5A to 5D show a summary statistic of the aortic blood pressure at the level of the ascending aorta of all virtual subjects. More specifically, the figures show the distribution, mean value and standard deviation in (mmHg) of: (a) mean blood pressure (MBP) in FIG. 5A, (b) pulse pressure (PP) in FIG. 5B, (c) diastolic blood pressure (DBP) in FIG. 5C, and (d) systolic blood pressure (SBP) in FIG. 5D, at the level of the ascending aorta for the in-silico data-base. This database presents physiological values with well-balanced distributions. The Cardiac outputs vary between 3.5 and 7.21/min, depending on the values of the heart rate (53, 63, and 72 beats/min) and stroke volume (66, 83, and 100 ml).

Before testing the model, it has to be calibrated. For both classical WK and the novel fractional-order models FWK models, the total peripheral resistance Rp parameter was evaluated from the ratio of the mean pressure to the mean blood flow. The remaining parameters of the models are: Θ_{Z}_{₂}_{^{α}}={τ_{α}, α} for FWK2 and Θ_{Z}_{₃}_{^{α}}={Z_{C}, τ_{α}, α} for FWK3, Θ_{Z}_{₂}={C} for WK2 and Θ_{Z}_{₃}={Z_{C}, C} for WK3. These parameters were estimated by fitting the models to the in-silico data illustrated in FIGS. 5A to 5D. First, the in-silico aortic input impedance was calculated. The in-silico blood flow and pressure for a single cardiac cycle expressed in the time domain were converted to the frequency domain using discrete Fourier transform, then the input impedance is formulated as the ratio of the harmonic of the blood pressure to the flow. Then, the optimal model parameters are identified by solving an optimization problem given by,

$$\begin{matrix} {Θ}^{*} = arg { min }_{Θ} f ( Θ ) , & ( 26 ) \\ f ( Θ ) = \sqrt{\frac{{\sum }_{i = 1}^{N} \{ {[ Re ( {Z}_{[ i ]} ) - Re ( {\hat{Z}}_{[ i ]} ( Θ ) ) ]}^{2} + {[ Im ( {Z}_{[ i ]} ) - Im ( {\hat{Z}}_{[ i ]} ( Θ ) ) ]}^{2} \}}{N}} , & ( 27 )\end{matrix}$$

where Θ* is the optimal parameter Θ that minimizes the cost function of equation (27), which corresponds to the root mean square error (RMSE), Re and Im denote the real and imaginary parts of the in-silico aortic input impedance Z and modeled impedance \hat{Z} evaluated at a specific harmonic i, and N is the total number of harmonics taken into account.

Furthermore, to evaluate the goodness of the fitting, the deviation of the model modulus from the in-silico aortic input impedance modulus was calculated based on the following expression:

$${D}_{i} [ \% ] = {[ \frac{\vert {\hat{Z}}_{[ i ]} ( Θ ) \vert - \vert {Z}_{[ i ]} \vert}{\vert {Z}_{[ i ]} \vert} ]}_{i = 1 …N} 100 \% . \qquad [ 28 ]$$

For ease of visualization of the comparison between all the models, for each virtual subject, the mean of D [%] was evaluated over the N harmonics based on equation:

$$Deviation [ \% ] = \frac{{\sum }_{i = 1}^{N} {D}_{i} [ \% ]}{N} . \qquad ( 29 )$$

Additionally, the normalized mean square error (NMSE) has been used to evaluate the goodness of fitting of the phase angle using the equation:

$$N M S E = 1 - \frac{{‖ ∠Z - ∠ \hat{Z} ( Θ ) ‖}^{2}}{{‖ ∠Z - mean ∠Z ‖}^{2}} , \qquad ( 30 )$$

where ∥.∥² indicates the 2-norm of a vector. The NMSE varies between −inf (bad fit) to 1 (perfect fit).

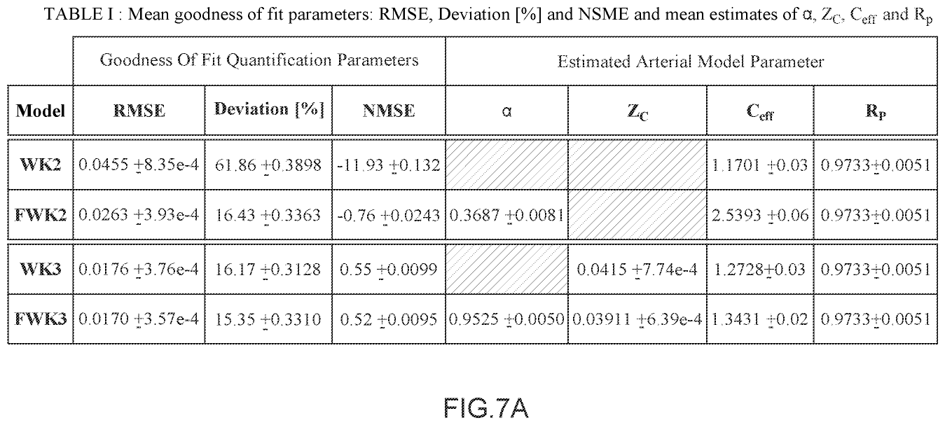

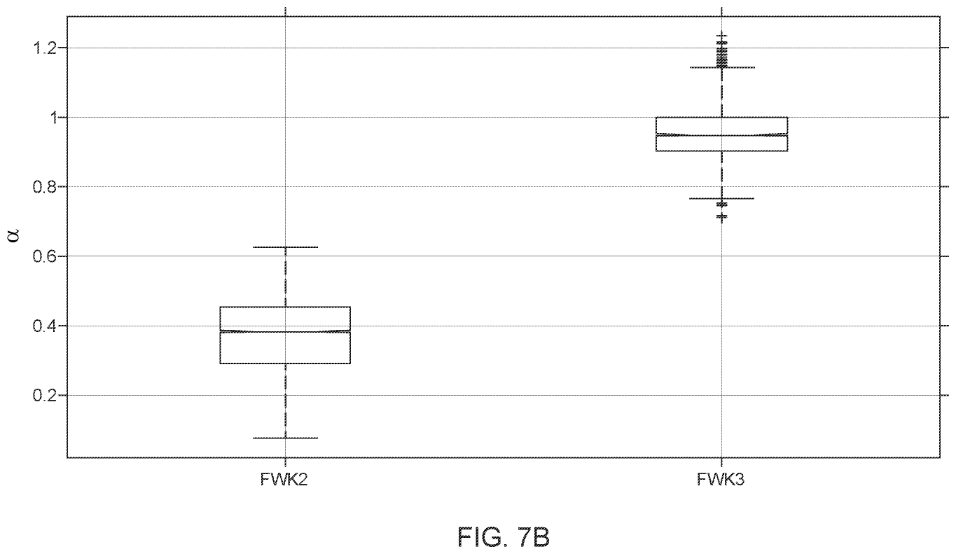

Next, the proposed novel models FWK2 and FWK3 were tested with the in-silico data and compared with the results of the traditional WK2 and WK3 models. The parameters of all these models have been determined numerically using an appropriate optimization technique implemented in MATLAB, based on the cost function defined in equation (26). Table 1, illustrated in FIG. 7A, shows the mean of RMSE, Deviation [%] and NMSE as quantifiers of the goodness of the overall model, modulus and phase angles fit, and the mean estimates of the arterial parameters.

FIG. 7B shows the distribution of the estimated fractional differentiation order a after fitting the proposed fractional-order impedance models FWK2 and FWK3 respectively, to the in-silico aortic input impedances evaluated at the level of the ascending aorta for 3,325 virtual subjects. These results show that, in most of the subjects, the values of a are different from the integer order (one) that corresponds to the order of classical WK models. With respect to FWK2, a is never equal to one, and for all the virtual subjects, its mean estimate value is about 0.3687±0.0081. With respect to FWK3, the mean of the estimated values of a is about 0.9525±0.005. Additionally, for some subjects, the estimated value of a is above one. These results confirm the assertion that the arterial systemic system involves viscoelastic behavior rather than pure elastic. Indeed, the fact that α≠1 indicates that the FoC element incorporates both resistive and capacitive quantities as demonstrated mathematically in equation (25).

Furthermore, these results justify the concept that the arterial system involves fractional-order behavior. In the proposed novel models, the fractional element, C_{α}, combines both resistive and capacitive properties which displays the viscoelastic behavior of the arterial vessel. The contribution of both properties is controlled by the fractional differentiation order (α) enabling more flexible physiological description. In fact, as a goes from one to zero, the resistive part of the FoC increases. On another hand, for the FWK3 model, a small resistance was added characterizing the characteristic aortic impedance (Z_{C}) comparing to FWK2. Hence, the values of a increase towards one (i.e., the resistive part of FoC decreases) as a counteraction of the contribution of Z_{C}. Indeed, in the case of FWK2, the resistive part of the FoC represents the whole resistance of the systemic system. However, for the FWK3, this quantity is shared between the two lumped elements Z_{C} and FoC (C_{α}).

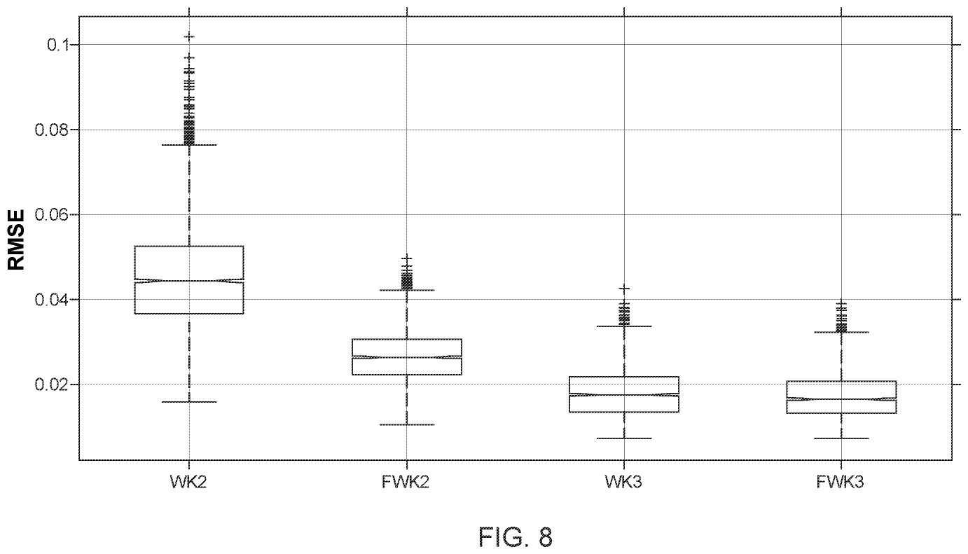

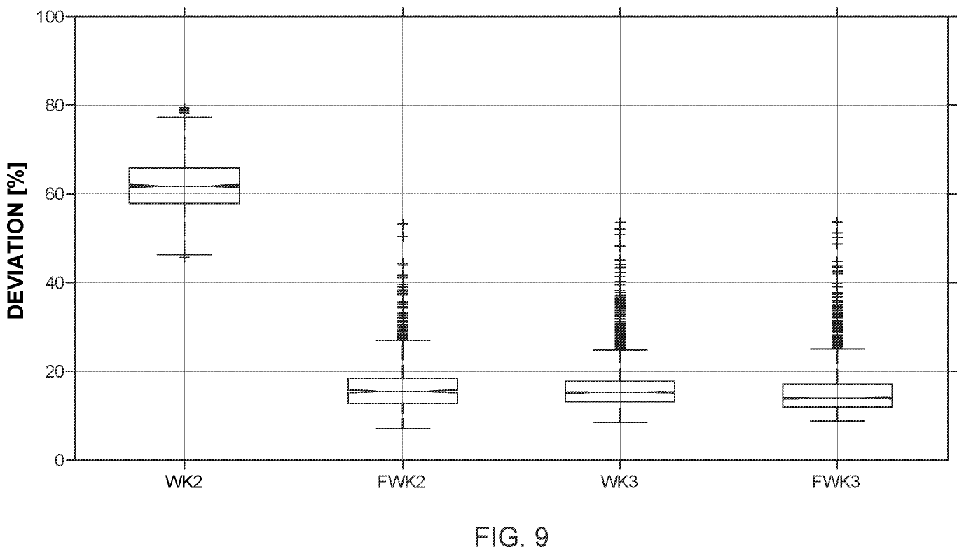

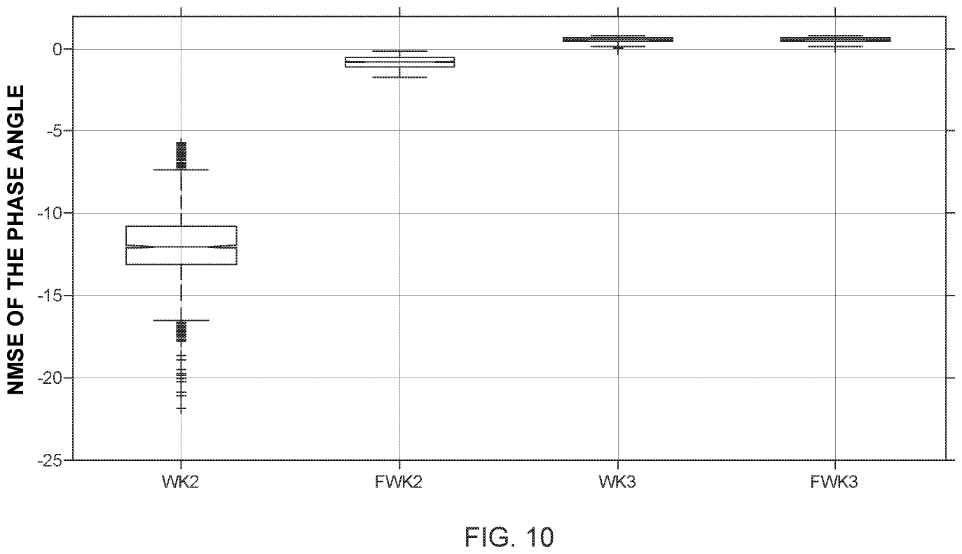

FIGS. 8 to 10 illustrate the comparison of the goodness of the fit between the proposed models (FWK2 and FWK3) and the standard WK models (WK2 and WK3) quantified respectively as: 1) RMSE for the overall models (FIG. 8), 2) Deviation[%] to evaluate the deviation of the modulus based model from the in-silico aortic input impedance modulus (FIG. 9), and 3) the NMSE to quantify the error of the phase angle fit (FIG. 10). Analyzing these figures and Table I, it can be seen that for all the subjects, WK2 has the highest RMSE and Deviation[%]. Furthermore, its (NMSE) is very low, approaching (−∞). Its mean value is equal to −11.93±0.132, which indicates a bad fitting of the phase angle. These findings are in agreement with the results reported in the open literature review. In fact, the decrease of the WK2 complex modulus to a negligible value and its phase angle to −90° at medium and high frequencies is in contrast to those measured in-vivo, where the modulus reduces to a plateau value and the phase angle tends to 0°.

By substituting the ideal capacitor with FoC in FWK2, the fitting of the aortic input impedance was sharply improved as can be noticed from the mean RMSE that decreases by almost one-half from WK2 to FWK2 and the Deviation reaches 16.43±0.33 versus 61.86±0.38 for WK2. Though the NMSE of FWK2 is not close to one (i.e., a good fit), the phase angle pattern has been enhanced comparing to the WK2. In addition, in terms of the modulus fitting, the FWK2 model is almost yielding the same performance as the WK3 model, whose Deviation is equal to 16.17±0.31.

The proposed FWK3 model provides the best fitting, but it is overall comparable to the WK3 model. Thus, these results confirm the reliability of both the WK3 as well as the FWK2 models in overcoming the limitations of the WK2 model, in describing the real input impedance. However, the use of the fractional-order element offer a proper measure for the better physiological analysis of the arterial function. In fact, an ideal capacitor is considered as a pure storage element that can only imitate pure elastic behavior rather than viscoelastic ones. On the other side, the fractional-order capacitor lumps both resistive and capacitive properties in one element allowing for a reduced-order description of the arterial viscoelastic characteristic. The proposed fractional-order model smoothly incorporates the complex effects and multi-scale properties of vascular tissues using a reduced-order configuration.

To have a fair comparison between the estimated compliance of the proposed fractional-order model and its corresponding standard Windkessel model, the effective compliance has been calculated for both models. The effective compliance can be derived as follows. For the fractional-order two element Windkessel model FWK2, the fractional aortic input impedance in the frequency domain is given by:

$$\begin{matrix}{Z}_{2}^{α} ( s ) = \frac{{R}_{p}}{1 + {( {τ}_{α} s )}^{α}} , & ( 31 ) \\ where & \\ {τ}_{α} = \sqrt[α]{{R}_{p} {C}_{α}} . & ( 32 )\end{matrix}$$

Based on equation (32), and from the estimated value of τₐ, the compliance C_{α} is given by:

$${C}_{α} = \frac{{( {τ}_{α} )}^{α}}{{R}_{p}} , \qquad ( 33 )$$

and then, the effective capacitance representing the effective compliance C_{eff} is given by:

$${C}_{eff} = {C}_{α} sin ( a \frac{π}{2} ) . \qquad ( 34 )$$

Note that by substituting a by one in equation (34), C_{eff} will represent the ideal capacitance that corresponds to the standard Windkessel compliance.

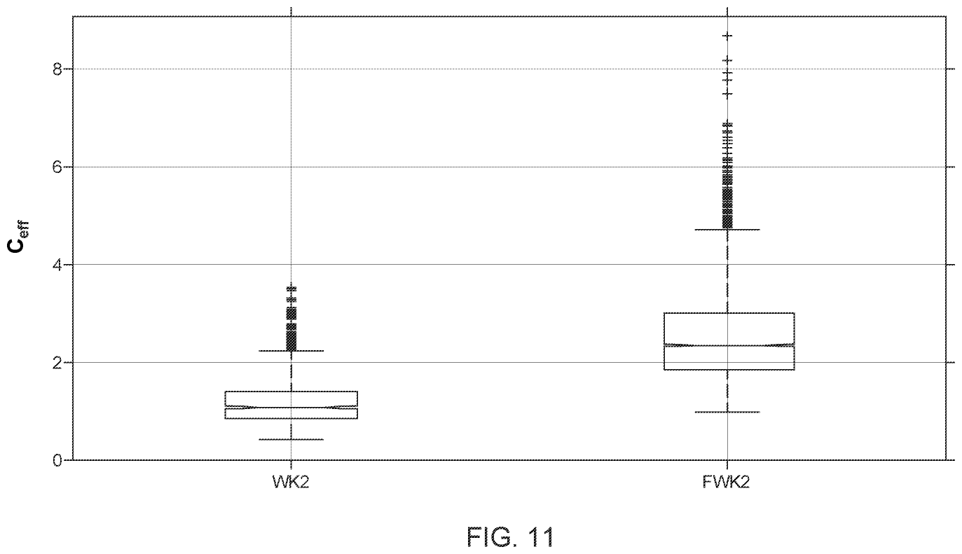

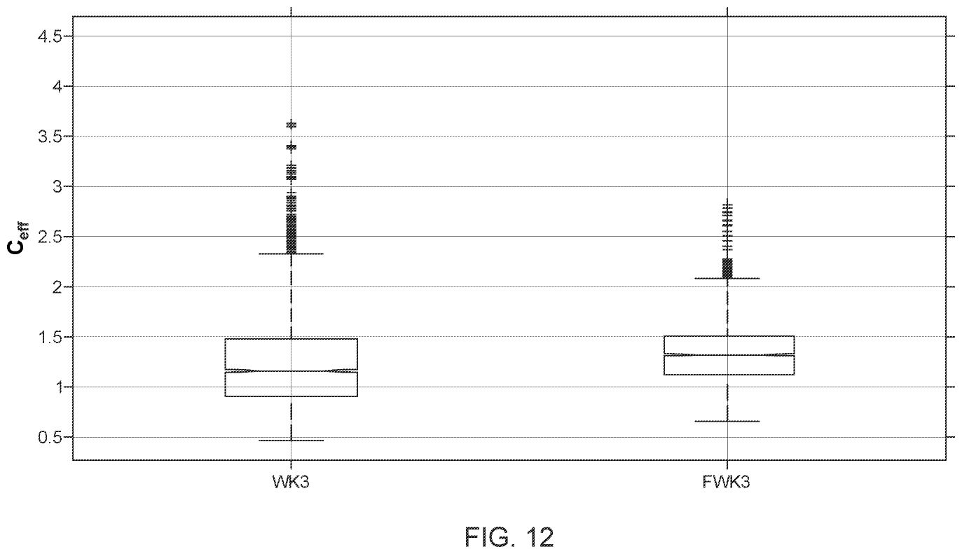

The distribution of the estimated effective compliance for WK2 and FWK2 are shown in FIG. 11 and for WK3 and FWK3 are shown in FIG. 12. It can be seen from FIG. 11 that C_{eff} of FWK2 is larger than C_{eff} of WK2. However, in FIG. 12, C_{eff} of FWK3 is close to C_{eff} of WK3. This result might be explained by the fact that, in the case of the FWK2 model, the value of a is less than 0.5, which signifies that the fractional-order element is more resistive than capacitive. Hence, the increase in the effective compliance might be interpretable as a compensation of the decrease of the capacitive part introduced by FoC. However, in the case of FWK3, the value of a is close to 1, which induces a reasonable C_{eff} between WK3 and FWK3.

FIG. 13 shows a comparative illustration of the estimated characteristic impedance Z_{C} of both WK3 and FWK3 models. It can be seen from these results that in both cases, the estimated values are almost the same. In general, the effect of adding Z_{C} to the FWK2 model configuration can be mostly visualized in the correction of the phase angle pattern only. However, for the case of the WK2 model, it affects both the phase angle and the modulus.

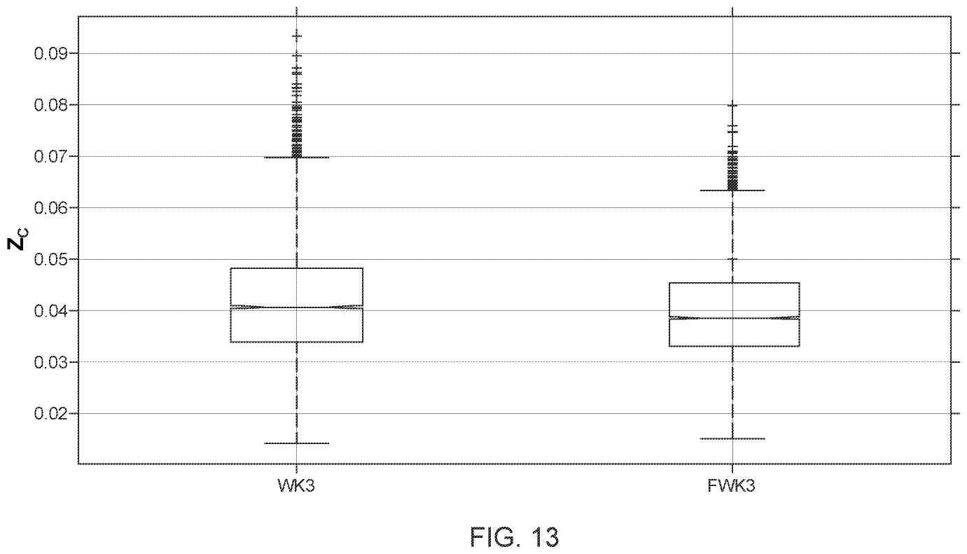

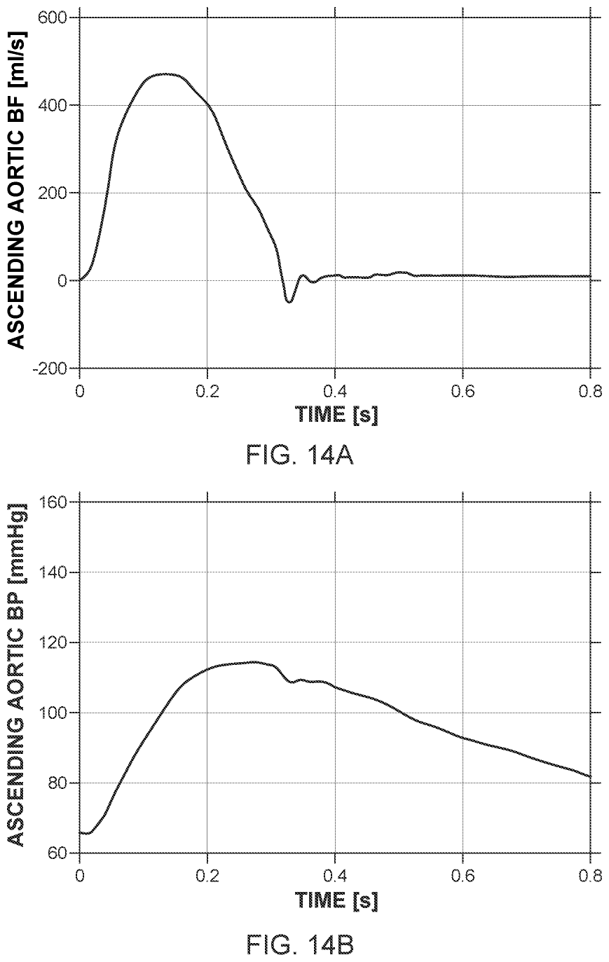

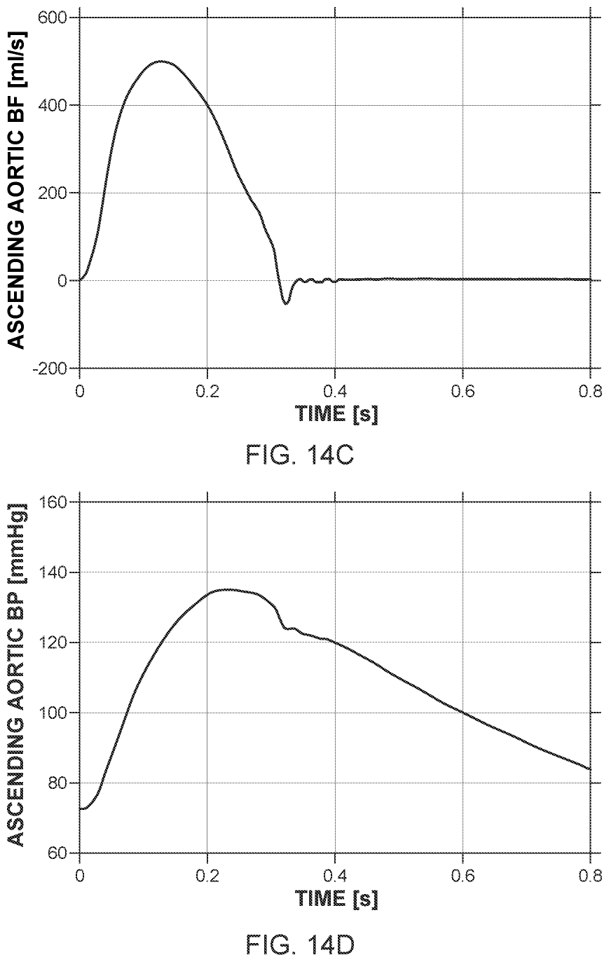

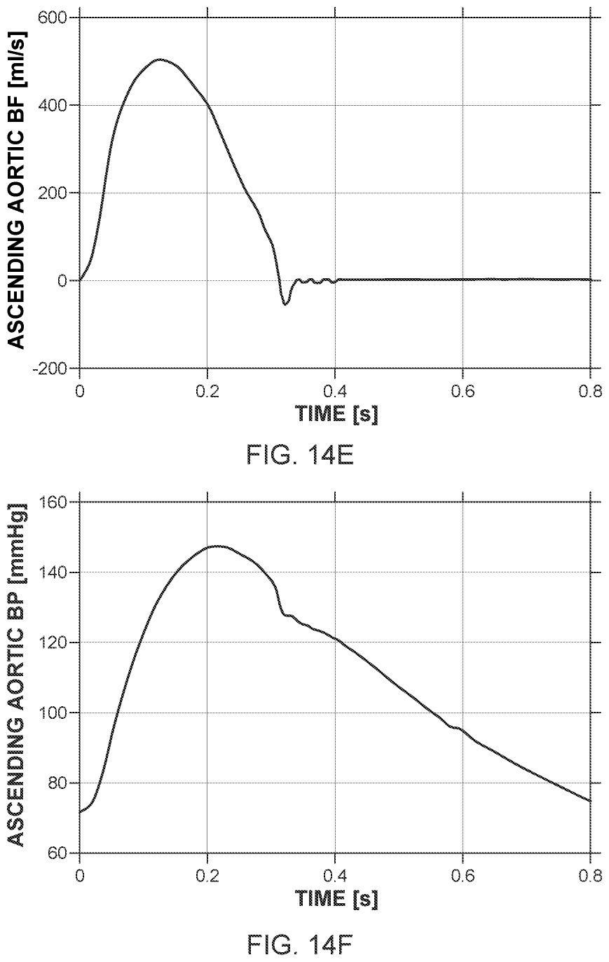

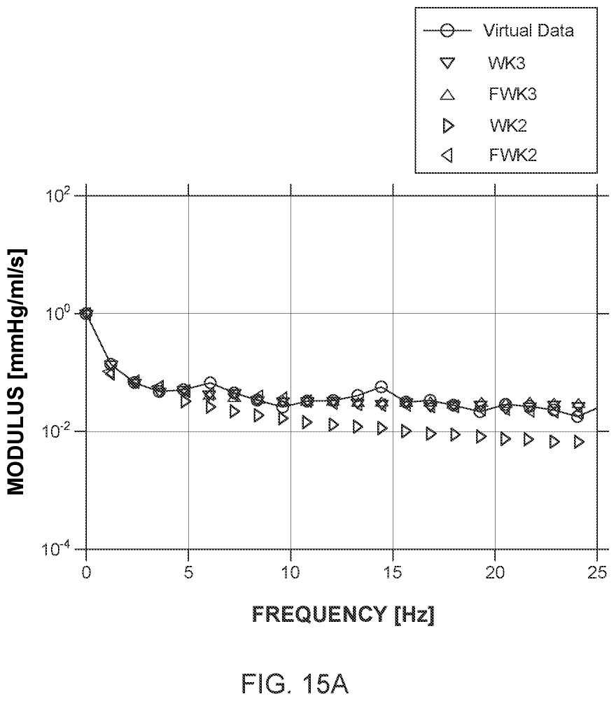

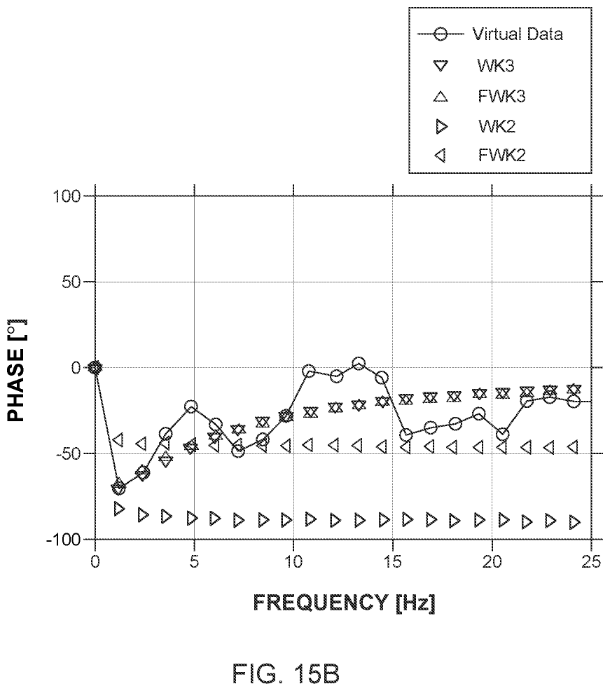

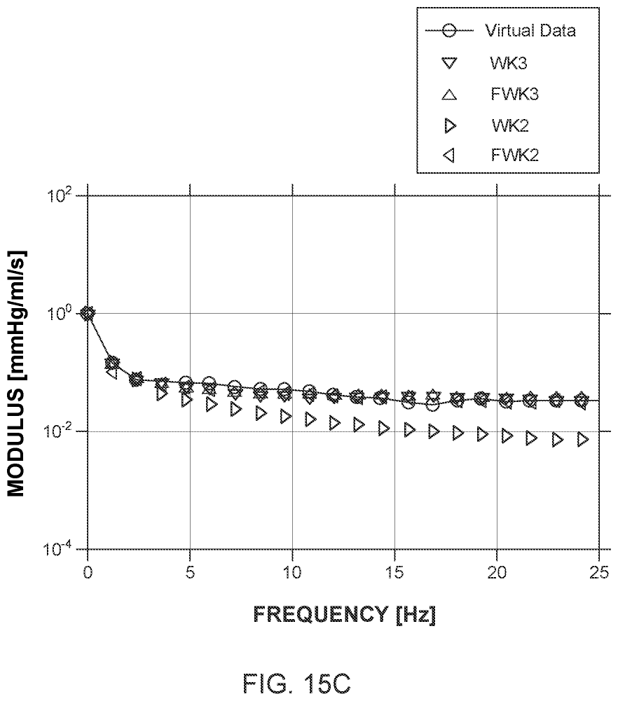

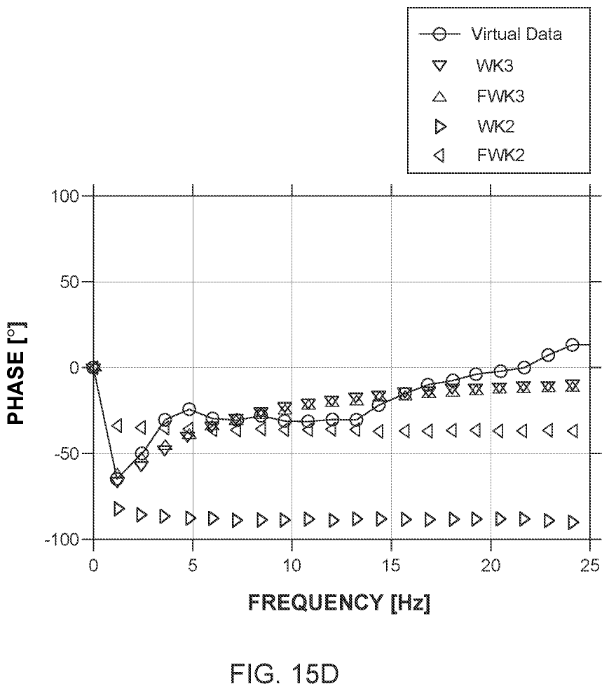

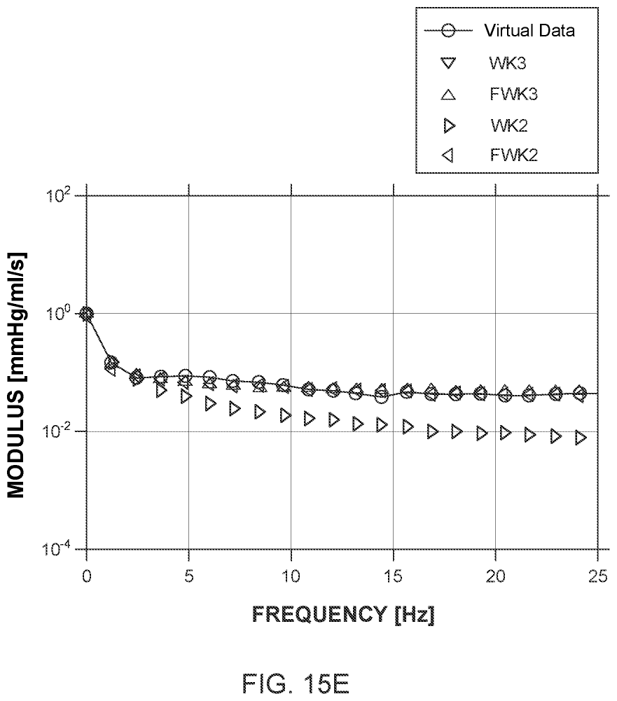

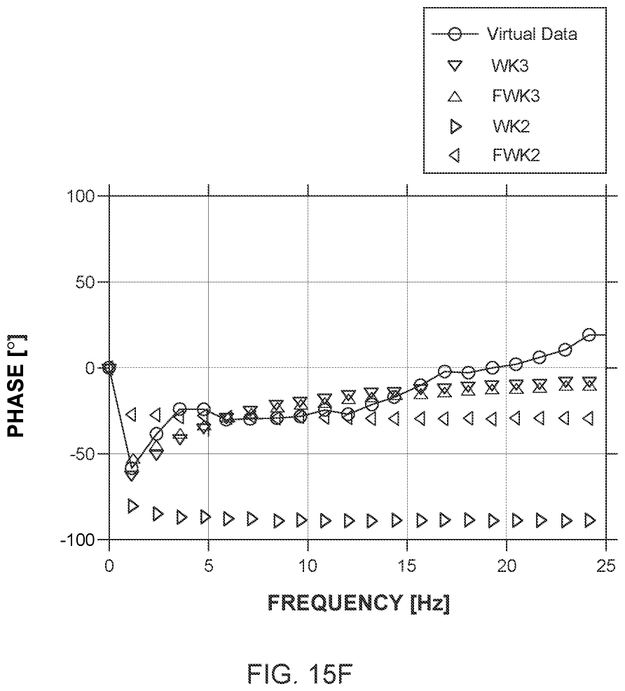

FIGS. 14A to 14F show the in-silico aortic input impedance patterns for different physiological states (normotensive in FIGS. 14A and 14B, hypertensive in FIGS. 14C and 14D, and severe-hypertensive in FIGS. 14E and 14F) with their corresponding aortic blood pressure and flow waveforms. FIGS. 15A to 15F illustrate the bar graphs of the hemodynamic parameters as well as the estimated parameters of both the proposed models and the standard Windkessel models. These results are in conformity with the conclusions afore-mentioned. In fact, the bar graphs in FIGS. 15A to 15F show that the FWK2 model is yielding to a good improvement in the prediction of the in-silico input impedance modulus and it is performing closely to WK3 and FWK3.

Furthermore, by checking the variation of a values of both the FWK2 and FWK3 models, from a physiological state to another, it can be noticed that the differentiation order a of the fractional-order operator is correlated with all the arterial parameter. For instance, the (α) values and the systolic blood pressure (SBP) or aortic pulse pressure (APP) show a negative correlation: from normotensive state to severe-hypertensive, SBP increases, however a for both FWK3 and FWK3 decreases. Hence, the fractional differentiation order might implicate a physiological insight. In fact, the decrease of a signifies an increase in the resistive part. On the other side, physiologically, one of the acute causes of high blood pressure is arterial stiffness. Accordingly, the new parameter α can be considered as a bio-marker that can lump the overall viscoelasticity properties of the human arterial tree. It might also have a potential role in enhancing the understanding of arterial stiffness dependencies.

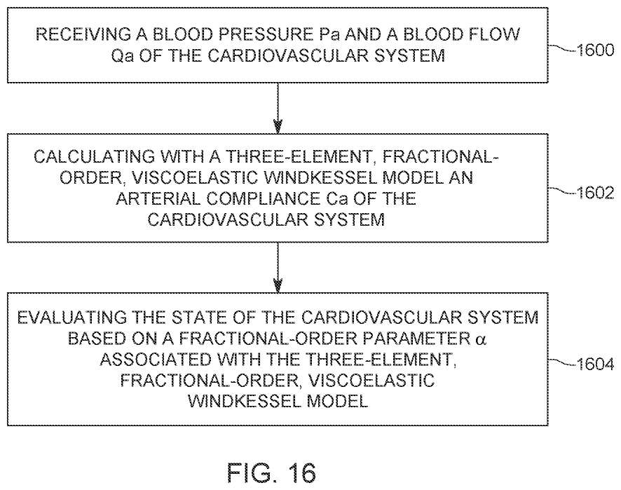

A method for assessing a state of a cardiovascular system is now discussed with regard to FIG. 16. The method includes a step 1600 of receiving a blood pressure Pa and a blood flow Qa of the cardiovascular system, a step 1602 of calculating, with a three-element, fractional-order, viscoelastic Windkessel model an arterial compliance C_{α} of the cardiovascular system, and a step 1604 of evaluating the state of the cardiovascular system based on a fractional-order parameter α associated with the three-element, fractional-order, viscoelastic Windkessel model.

The method may also include a step of calculating an aortic input impedance Zᵢₙ of the cardiovascular system as a ratio of (1) the blood pressure Pa and (2) the blood flow Qa. In one application, the blood pressure Pa is estimated at the aorta, and the blood flow Qa is estimated at the aorta. The three-element, fractional-order, viscoelastic Windkessel model includes an aortic specific impedance Z_{c}, a fractional-order capacitor C_{F}, and an arterial peripheral resistance Rₚ. The fractional-order capacitor C_{F} and the arterial peripheral resistance Rₚ are connected in parallel, and the aortic specific impedance Z_{c} is connected in series with a block formed by the fractional-order capacitor C_{F} and the arterial peripheral resistance Rₚ. The fractional-order capacitor C_{F} is a constant phase element characterized by the fractional-order parameter α.

The method may also include calculating a fractional-order impedance Z_{F} of the fractional-order capacitor C_{F}, where the arterial compliance C_{α} of the cardiovascular system is the fractional-order impedance Z_{F} of the fractional-order capacitor C_{F}, and/or calculating the Young modulus E of arteries of the cardiovascular system as an inverse of the arterial compliance C_{α}, and/or estimating an arterial stiffness of the cardiovascular system based on the Young modulus E, and/or determining whether a patient is hypotensive, hypertensive, or normotensive based on the fractional-order parameter α.

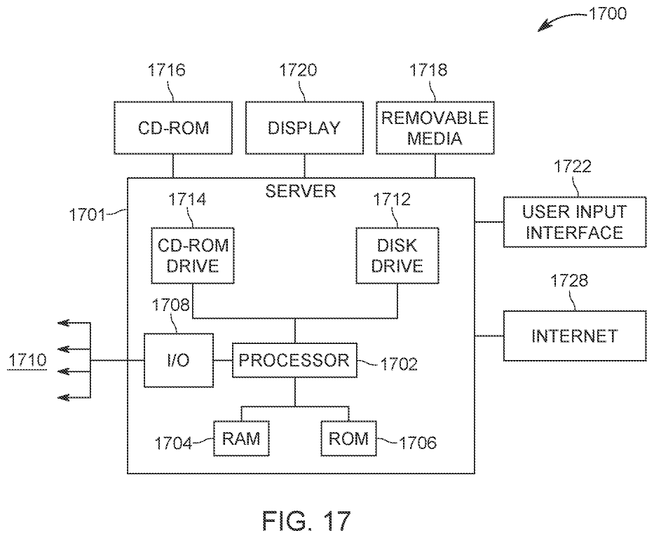

The above-discussed procedures and methods may be implemented in a computing device as illustrated in FIG. 17. Hardware, firmware, software or a combination thereof may be used to perform the various steps and operations described herein.

Computing device 1700 suitable for performing the activities described in the embodiments discussed above may include a server 1701. Such a server 1701 may include a central processor (CPU) 1702 coupled to a random access memory (RAM) 1704 and to a read-only memory (ROM) 1706. ROM 1706 may also be other types of storage media to store programs, such as programmable ROM (PROM), erasable PROM (EPROM), etc. Processor 1702 may communicate with other internal and external components through input/output (I/O) circuitry 1708 and bussing 1710 to provide control signals and the like. Processor 1702 carries out a variety of functions as are known in the art, as dictated by software and/or firmware instructions.

Server 1701 may also include one or more data storage devices, including hard drives 1712, CD-ROM drives 1714 and other hardware capable of reading and/or storing information, such as DVD, etc. In one embodiment, software for carrying out the above-discussed steps may be stored and distributed on a CD-ROM or DVD 1716, a USB storage device 1718 or other form of media capable of portably storing information. These storage media may be inserted into, and read by, devices such as CD-ROM drive 1714, disk drive 1712, etc. Server 1701 may be coupled to a display 1720, which may be any type of known display or presentation screen, such as LCD, plasma display, cathode ray tube (CRT), etc. A user input interface 1722 is provided, including one or more user interface mechanisms such as a mouse, keyboard, microphone, touchpad, touch screen, voice-recognition system, etc.

Server 1701 may be coupled to other devices, such as medical instruments, detectors, sensors, etc. The server may be part of a larger network configuration as in a global area network (GAN) such as the Internet 1728, which allows ultimate connection to various landline and/or mobile computing devices.

The disclosed embodiments provide a method and system that is capable to assess the cardiovascular system using a two- or three-element fractional-order viscoelastic Windkessel model. The embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] T. C. Doehring, A. D. Freed, E. O. Carew, and I. Vesely, “Fractional order viscoelasticity of the aortic valve cusp: an alternative to quasi-linear viscoelasticity,” Journal of biomechanical engineering, vol. 127, no. 4, pp. 700-708, 2005.
[2] D. Craiem and R. L. Armentano, “A fractional derivative model to describe arterial viscoelasticity,” Biorheology, vol. 44, no. 4, pp. 251-263, 2007.
[3] D. Craiem, F. Rojo, J. Atienza, G. Guinea, and R. L. Armentano, “Fractional calculus applied to model arterial viscoelasticity,” Latin American applied research, vol. 38, no. 2, pp. 141-145, 2008.
[4] D. Craiem, F. J. Rojo, J. M. Atienza, R. L. Armentano, and G. V. Guinea, “Fractional-order viscoelasticity applied to describe uniaxial stress relaxation of human arteries,” Physics in medicine and biology, vol. 53, no. 17, p. 4543, 2008.
[5] J. P. Zerpa, A. Canelas, B. Sensale, D. B. Santana, and R. Armentano, “Modeling the arterial wall mechanics using a novel high-order viscoelastic fractional element,” Applied Mathematical Modelling, vol. 39, no. 16, pp. 4767-4780, 2015.

1. A method for assessing a state of a cardiovascular system, the method comprising:
receiving a blood pressure Pa and a blood flow Qa of the cardiovascular system;
calculating with a fractional-order, viscoelastic Windkessel model an arterial compliance C_{α} of the cardiovascular system; and
evaluating the state of the cardiovascular system based on a fractional-order parameter α associated with the fractional-order, viscoelastic Windkessel model.

2. The method of claim 1, further comprising:
calculating an aortic input impedance Zᵢₙ of the cardiovascular system as a ratio of (1) the blood pressure Pa and (2) the blood flow Qa.

3. The method of claim 2, wherein the blood pressure Pa and the blood flow Qa are estimated at the aorta.

4. The method of claim 3, wherein the fractional-order, viscoelastic Windkessel model is defined by three parameters, a specific impedance Z_{c}, a fractional-order capacitor C_{F}, and an arterial peripheral resistance Rₚ.

5. The method of claim 4, wherein the fractional-order capacitor C_{F} and the arterial peripheral resistance Rₚ are connected in parallel to each other, and the specific impedance Z_{c} is connected in series with a block formed by the fractional-order capacitor C_{F} and the arterial peripheral resistance Rₚ.

6. The method of claim 5, wherein the fractional-order capacitor C_{F} is a constant phase element characterized by the fractional-order parameter α.

7. The method of claim 6, further comprising:
calculating a fractional-order impedance Z_{F} of the fractional-order capacitor C_{F}.

8. The method of claim 7, wherein the arterial compliance C_{α} of the cardiovascular system is the fractional-order impedance Z_{F} of the fractional-order capacitor C_{F}.

9. The method of claim 8, further comprising:
calculating the Young modulus E of arteries of the cardiovascular system as an inverse of the arterial compliance C_{α}.

10. The method of claim 9, further comprising:
estimating an arterial stiffness of the cardiovascular system based on the Young modulus E.

11. The method of claim 1, further comprising:
determining whether a patient is hypotensive, hypertensive, or normotensive based on the fractional-order parameter α.

12. A computing device for assessing a state of a cardiovascular system, the computing device comprising:
an interface for receiving a blood pressure Pa and a blood flow Qa of the cardiovascular system; and
a processor connected to the interface and configured to,
calculate with a fractional-order, viscoelastic, Windkessel model an arterial compliance C_{α} of the cardiovascular system; and
evaluate the state of the cardiovascular system based on a fractional-order parameter α associated with the fractional-order, viscoelastic Windkessel model.

13. The computing device of claim 12, wherein the processor is further configured to:
calculate an aortic input impedance Zᵢₙ of the cardiovascular system as a ratio of (1) the blood pressure Pa and (2) the blood flow Qa.

14. The computing device of claim 13, wherein the blood pressure Pa and the blood flow Qa is estimated at the aorta, and
wherein the fractional-order, viscoelastic, Windkessel model includes an aortic specific impedance Z_{c}, a fractional-order capacitor C_{F}, and an arterial peripheral resistance Rₚ.

15. The computing device of claim 14, wherein the fractional-order capacitor C_{F} and the arterial peripheral resistance Rₚ are connected in parallel, and the aortic specific impedance Z_{c} is connected in series with a block formed by the fractional-order capacitor C_{F} and the arterial peripheral resistance Rₚ.

16. The computing device of claim 15, wherein the fractional-order capacitor C_{F} is a constant phase element characterized by the fractional-order parameter α.

17. The computing device of claim 16, wherein the processor is further configured to:
calculate a fractional-order impedance Z_{F} of the fractional-order capacitor C_{F}, wherein the arterial compliance C_{α} of the cardiovascular system is the fractional-order impedance Z_{F} of the fractional-order capacitor C_{F};
calculate the Young modulus E of arteries of the cardiovascular system as an inverse of the arterial compliance C_{α};
estimate an arterial stiffness of the cardiovascular system based on the Young modulus E; and
determine whether a patient is hypotensive, hypertensive, or normotensive based on the fractional-order parameter α.

18. A non-transitory computer readable medium including computer executable instructions, wherein the instructions, when executed by a processor, implement instructions for assessing a state of a cardiovascular system, the instructions comprising:
receiving a blood pressure Pa and a blood flow Qa of the cardiovascular system;
calculating with a fractional-order, viscoelastic Windkessel model an arterial compliance C_{α} of the cardiovascular system; and
evaluating the state of the cardiovascular system based on a fractional-order parameter α associated with the fractional-order, viscoelastic Windkessel model.

19. The medium of claim 18, further comprising:
calculating an aortic input impedance Zᵢₙ of the cardiovascular system as a ratio of (1) the blood pressure Pa and (2) the blood flow Qa,
wherein the blood pressure Pa and the blood flow Qa are estimated at the aorta, and
wherein the fractional-order, viscoelastic, Windkessel model includes an aortic specific impedance Z_{c}, a fractional-order capacitor C_{F}, and an arterial peripheral resistance Rₚ.

20. The medium of claim 19, wherein the fractional-order capacitor C_{F} and the arterial peripheral resistance Rₚ are connected in parallel, and the aortic specific impedance Z_{c} is connected in series with a block formed by the fractional-order capacitor C_{F} and the arterial peripheral resistance Rₚ, and wherein the fractional-order capacitor C_{F} is a constant phase element characterized by the fractional-order parameter α.

21. The medium of claim 20, further comprising:
calculating a fractional-order impedance Z_{F} of the fractional-order capacitor C_{F}, wherein the arterial compliance C_{α} of the cardiovascular system is the fractional-order impedance Z_{F} of the fractional-order capacitor C_{F};
calculating the Young modulus E of arteries of the cardiovascular system as an inverse of the arterial compliance C_{α};
estimating an arterial stiffness of the cardiovascular system based on the Young modulus E; and
determining whether a patient is hypotensive, hypertensive, or normotensive based on the fractional-order parameter α.
